(19) United States
(12) Patent Application Publication
(10) Pub. No.: US 2026/0284138 A1
(43) Pub. Date: Sep. 24, 2026

(54) METHODS OF MODULATING THE EPITHELIAL-MESENCHYMAL TRANSITION

(71) Applicant: RS Oncology, LLC, Cambridge, MA (US)

(72) Inventor: Brian Cunniff, Jericho, VT (US)

(21) Appl. No.: 19/475,255

(22) PCT Filed: Apr. 5, 2024
(86) PCT No.: PCT/US2024/023329

§ 371 Date: Oct. 14, 2025

Related U.S. Application Data
(60) Provisional application No. 63/459,072, filed on Apr. 13, 2023.
(60) Provisional application No. 63/537,368, filed on Sep. 8, 2023.

(51) Int. Cl. A61K 38/12; A61K 31/40; A61K 31/428; A61K 31/44; A61K 31/517; A61K 45/06; A61P 35/00; C12Q 1/6886

(52) U.S. Cl. CPC A61K 38/12; A61K 31/40; A61K 31/428; A61K 31/44; A61K 31/517; A61K 45/06; A61P 35/00; C12Q 1/6886; C12Q 2600/106

(57) ABSTRACT

Disclosed are methods of treating cancer in a subject in need thereof. The method may comprise administering to the subject an effective amount of a PRX3 inhibitor. The method may comprise determining quantitative expression of a biomark-er associated with epithelial-mesenchymal transition (EMT) and administering to the subject an effective amount of a PRX3 inhibitor if the biomarker characteristic for a mesenchymal cell phenotype is increased or if the biomarker characteristic for an ep-ithelial cell phenotype is decreased.

### RELATED APPLICATIONS

This patent application claims priority to U.S. Provisional Patent Application No. 63/459,072, filed Apr. 13, 2023, and U.S. Provisional Patent Application No. 63/537,368, filed Sep. 8, 2023, each of which is hereby incorporated by reference in its entirety.

### BACKGROUND

In the U.S., cancer is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult. Although tremendous advances have been made in chemotherapy, currently available therapeutics and therapies remain unsatisfactory and the prognosis for the majority of patients diagnosed with cancer remains poor. Often, the applicability and/or effectiveness of chemotherapy, as well as other therapies and diagnostics employing potentially toxic moieties, is limited by undesired side effects. Accordingly, there is a great need to identify effective agents in treating cancer.

### SUMMARY OF THE INVENTION

In certain aspects, described herein are methods of treating cancer in a subject in need thereof comprising administering to the subject an effective amount of a PRX3 inhibitor. Numerous embodiments are further provided that can be applied to any aspect of the present invention described herein. In some embodiments, the cancer is selected from the group consisting of sarcomatoid mesotheliomas(S), epithelioid mesotheliomas (E), sarcoma, sarcomatoid carcinoma, thymoma, adrenocortical cancer, uterine carcinosarcoma, uterine corpus endometrioid carcinoma, kidney clear cell carcinoma, brain lower grade glioma, pheochromocytoma and paraganglioma, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, KRAS-mutant melanoma, renal cell carcinoma of kidney, kidney papillary cell carcinoma, reserve cell carcinoma, triple-negative breast cancer, primary thrombocytosis, primary macroglobulinemia, small cell lung cancer, ovarian serous cystadenocarcinoma, primary brain tumors, malignant pancreatic insulinoma, malignant carcinoid, premalignant skin lesions, prostate neuroendocrine cancer, testicular germ cell tumor, lymphomas, genitourinary tract cancer, malignant hypercalcemia, endometrial cancer, adrenal cortical cancer, Harding-Passey melanoma, juvenile melanoma, lentigo maligna melanoma, acral-lentiginous melanoma, amelanotic melanoma, benign juvenile melanoma, Cloudman's melanoma, S91 melanoma, nodular melanoma, subungual melanoma, and superficial spreading melanoma.

In some embodiments, the PRX3 inhibitor is thiostrepton. In some embodiments, the cancer is a sarcoma. In some embodiments, the sarcoma is angiosarcoma, dermatofibrosarcoma, epithelioid sarcoma, Ewing's sarcoma, fibrosarcoma, gastrointestinal stromal tumors (GIST), Kaposi sarcoma, leiomyosarcoma, liposarcomas, dedifferentiated liposarcoma, pleomorphic myxoid liposarcoma, malignant fibrous histiocytoma, undifferentiated pleomorphic sarcoma, neurofibrosarcoma, rhabdomyosarcoma (RMS), embryonal rhabdomyosarcoma, alveolar rhabdomyosarcoma, synovial sarcoma, alveolar soft part sarcoma, chondrosarcoma, desmoid sarcoma, malignant peripheral nerve sheath tumor (MPNST), uterine carcinosarcoma, myxofibrosarcoma, undifferentiated sarcoma NOS, INI-deficient soft tissue sarcoma NOS, malignant peripheral nerve sheath tumor, desmoplastic small round cell tumor, spindle cell/sclerosing rhabdomyosarcoma, undifferentiated spindle cell sarcoma, atypical teratoid/rhabdoid tumor, fibromatosis, inflammatory myofibroblastic tumor, infantile fibrosarcoma, sclerosing epithelioid fibrosarcoma, myofibromatosis, undifferentiated hepatic sarcoma, endometrial stromal sarcoma, lipoblastomatosis, sarcomatoid disease, melanocytic clear cell sarcoma (such as soft tissue melanoma), dermal pleomorphic sarcoma, or osteosarcoma. In some embodiments, the cancer is a sarcomatoid carcinoma. In some embodiments, the sarcomatoid carcinoma is associated with the subject's skin, bone, mouth, thyroid gland, breast, stomach, small intestine, liver, pancreas, uterus, prostate, bladder, kidneys, mediastinum, or lung.

In some embodiments, the method further comprises administering an effective amount of a cancer chemotherapeutic agent. In some embodiments, the cancer chemotherapeutic agent is selected from the group consisting of thiotepa, cyclophosphamide, busulfan, improsulfan, piposulfan, benzodopa, carboquone, meturedopa, uredopa, altretamine, triethylenemelamine, triethylenephosphoramide, triethylenethiophosphoramide, trimethylolmelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin; neocarzinostatin chromophore, aclacinomycins, actinomycin, authramycin, azaserine, bleomycins, cactinomycin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatrexate, demecolcine, diaziquone, eflornithine, elliptinium acetate, epothilone, etoglucid, gallium nitrate, hydroxyurea, lentinan, lonidamine, maytansine, ansamitocins, mitoguazone, mitoxantrone, mopidamol, nitracrine, pentostatin, phenamet, pirarubicin, losoxantrone, podophyllinic acid, 2-ethylhydrazide, procarbazine, PSK polysaccharide complex, razoxane, rhizoxin, sizofiran, spirogermanium, tenuazonic acid, triaziquone; 2,2′,2″-trichlorotriethylamine, trichothecene, T-2 toxin, verrucarin A, roridin A, anguidine, urethane, vindesine, dacarbazine, mannomustine, mitobronitol, mitolactol, pipobroman, gacytosine, arabinoside, cyclophosphamide, paclitaxel, docetaxel, chlorambucil, gemcitabine, 6-thioguanine, mercaptopurine, methotrexate, cisplatin, oxaliplatin, carboplatin, vinblastine, platinum, etoposide, ifosfamide, vincristine, vinorelbine, novantrone, teniposide, edatrexate, daunomycin, aminopterin, xeloda, ibandronate, irinotecan, RFS 2000, difluoromethylornithine, retinoic acid, and capecitabine.

In some aspects, described herein are methods of treating cancer in a subject in need thereof comprising determining or receiving information on the quantitative expression of a biomarker associated with epithelial-mesenchymal transition (EMT), wherein the biomarker is characteristic for a mesenchymal cell phenotype or for an epithelial cell phenotype. The method may further comprise administering to the subject an effective amount of a PRX3 inhibitor if the biomarker characteristic for a mesenchymal cell phenotype is increased or if the biomarker characteristic for an epithelial cell phenotype is decreased. Numerous embodiments are further provided that can be applied to any aspect of the present invention described herein. In some embodiments, the PRX3 inhibitor is thiostrepton. In some embodiments, PRX3 inhibitor decreases the mesenchymal cell phenotype or increases the epithelial cell phenotype.

In some embodiments, the method further comprises: determining or receiving information on the quantitative expression of the biomarker associated with EMT after administering the PRX3 inhibitor, and administering an effective amount of a cancer chemotherapeutic agent if the biomarker characteristic for a mesenchymal cell phenotype is decreased or if the biomarker characteristic for an epithelial cell phenotype is increased. In some embodiments, the cancer chemotherapeutic agent is selected from the group consisting of thiotepa, cyclophosphamide, busulfan, improsulfan, piposulfan, benzodopa, carboquone, meturedopa, uredopa, altretamine, triethylenemelamine, triethylenephosphoramide, triethylenethiophosphoramide, trimethylolmelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin; neocarzinostatin chromophore, aclacinomycins, actinomycin, authramycin, azaserine, bleomycins, cactinomycin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatrexate, demecolcine, diaziquone, eflornithine, elliptinium acetate, epothilone, etoglucid, gallium nitrate, hydroxyurea, lentinan, lonidamine, maytansine, ansamitocins, mitoguazone, mitoxantrone, mopidamol, nitracrine, pentostatin, phenamet, pirarubicin, losoxantrone, podophyllinic acid, 2-ethylhydrazide, procarbazine, PSK polysaccharide complex, razoxane, rhizoxin, sizofiran, spirogermanium, tenuazonic acid, triaziquone; 2,2′,2″-trichlorotriethylamine, trichothecene, T-2 toxin, verrucarin A, roridin A, anguidine, urethane, vindesine, dacarbazine, mannomustine, mitobronitol, mitolactol, pipobroman, gacytosine, arabinoside, cyclophosphamide, paclitaxel, docetaxel, chlorambucil, gemcitabine, 6-thioguanine, mercaptopurine, methotrexate, cisplatin, oxaliplatin, carboplatin, vinblastine, platinum, etoposide, ifosfamide, vincristine, vinorelbine, novantrone, teniposide, edatrexate, daunomycin, aminopterin, xeloda, ibandronate, irinotecan, RFS 2000, difluoromethylornithine, retinoic acid, and capecitabine.

In some embodiments, the cancer is selected from the group consisting of sarcoma, sarcomatoid carcinoma, thymoma, glioblastoma multiforme, adrenocortical cancer, uterine carcinosarcoma, uterine corpus endometrioid carcinoma, kidney clear cell carcinoma, mesothelioma, brain lower grade glioma, pheochromocytoma and paraganglioma, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, KRAS-mutant melanoma, renal cell carcinoma of kidney, kidney papillary cell carcinoma, reserve cell carcinoma, Hodgkin's Disease, Non-Hodgkin's Lymphoma, multiple myeloma, neuroblastoma, breast cancer, triple-negative breast cancer, ovarian cancer, lung cancer, primary thrombocytosis, primary macroglobulinemia, small cell lung cancer, ovarian serous cystadenocarcinoma, primary brain tumors, stomach cancer, colon cancer, malignant pancreatic insulinoma, malignant carcinoid, premalignant skin lesions, testicular cancer, prostate neuroendocrine cancer, testicular germ cell tumor, lymphomas, thyroid carcinoma, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungual melanoma, and superficial spreading melanoma. In some embodiments, the mesothelioma is sarcomatoid mesotheliomas(S) or epithelioid mesotheliomas (E).

In some embodiments, the cancer is a sarcoma. In some embodiments, the sarcoma is angiosarcoma, dermatofibrosarcoma, epithelioid sarcoma, Ewing's sarcoma, fibrosarcoma, gastrointestinal stromal tumors (GIST), Kaposi sarcoma, leiomyosarcoma, liposarcomas, dedifferentiated liposarcoma, pleomorphic myxoid liposarcoma, malignant fibrous histiocytoma, undifferentiated pleomorphic sarcoma, neurofibrosarcoma, rhabdomyosarcoma (RMS), embryonal rhabdomyosarcoma, alveolar rhabdomyosarcoma, synovial sarcoma, alveolar soft part sarcoma, chondrosarcoma, desmoid sarcoma, malignant peripheral nerve sheath tumor (MPNST), uterine carcinosarcoma, myxofibrosarcoma, undifferentiated sarcoma NOS, INI-deficient soft tissue sarcoma NOS, malignant peripheral nerve sheath tumor, desmoplastic small round cell tumor, spindle cell/sclerosing rhabdomyosarcoma, undifferentiated spindle cell sarcoma, atypical teratoid/rhabdoid tumor, fibromatosis, inflammatory myofibroblastic tumor, infantile fibrosarcoma, sclerosing epithelioid fibrosarcoma, myofibromatosis, undifferentiated hepatic sarcoma, endometrial stromal sarcoma, lipoblastomatosis, sarcomatoid disease, melanocytic clear cell sarcoma (such as soft tissue melanoma), dermal pleomorphic sarcoma, or osteosarcoma.

In some embodiments, the cancer is a sarcomatoid carcinoma. In some embodiments, the sarcomatoid carcinoma is associated with the subject's skin, bone, mouth, thyroid gland, breast, stomach, small intestine, liver, pancreas, uterus, prostate, bladder, kidneys, mediastinum, or lung.

In some aspects, described herein are methods of treating cancer in a subject in need thereof. In some embodiments, the subject comprises administering to the subject an effective amount of a PRX3 inhibitor and a SLC7A11 inhibitor. In some embodiments, the PRX3 inhibitor is thiostrepton. In some embodiments, the PRX3 inhibitor decreases the mesenchymal cell phenotype or increases the epithelial cell phenotype. In some embodiments, the SLC7A11 inhibitor is riluzole, sulfasalazine, erastin, sorafenib, or lanperisone. In some embodiments, the method further comprises administering an effective amount of a cancer chemotherapeutic agent. In some embodiments, the cancer chemotherapeutic agent is selected from the group consisting of thiotepa, cyclophosphamide, busulfan, improsulfan, piposulfan, benzodopa, carboquone, meturedopa, uredopa, altretamine, triethylenemelamine, triethylenephosphoramide, triethylenethiophosphoramide, trimethylolmelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin; neocarzinostatin chromophore, aclacinomycins, actinomycin, authramycin, azaserine, bleomycins, cactinomycin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatrexate, demecolcine, diaziquone, eflornithine, elliptinium acetate, epothilone, etoglucid, gallium nitrate, hydroxyurea, lentinan, lonidamine, maytansine, ansamitocins, mitoguazone, mitoxantrone, mopidamol, nitracrine, pentostatin, phenamet, pirarubicin, losoxantrone, podophyllinic acid, 2-ethylhydrazide, procarbazine, PSK polysaccharide complex, razoxane, rhizoxin, sizofiran, spirogermanium, tenuazonic acid, triaziquone; 2,2′,2″-trichlorotriethylamine, trichothecene, T-2 toxin, verrucarin A, roridin A, anguidine, urethane, vindesine, dacarbazine, mannomustine, mitobronitol, mitolactol, pipobroman, gacytosine, arabinoside, cyclophosphamide, paclitaxel, docetaxel, chlorambucil, gemcitabine, 6-thioguanine, mercaptopurine, methotrexate, cisplatin, oxaliplatin, carboplatin, vinblastine, platinum, etoposide, ifosfamide, vincristine, vinorelbine, novantrone, teniposide, edatrexate, daunomycin, aminopterin, xeloda, ibandronate, irinotecan, RFS 2000, difluoromethylornithine, retinoic acid, and capecitabine.

In some embodiments, the cancer is selected from the group consisting of sarcoma, sarcomatoid carcinoma, thymoma, glioblastoma multiforme, adrenocortical cancer, uterine carcinosarcoma, uterine corpus endometrioid carcinoma, kidney clear cell carcinoma, mesothelioma, brain lower grade glioma, pheochromocytoma and paraganglioma, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, KRAS-mutant melanoma, renal cell carcinoma of kidney, kidney papillary cell carcinoma, reserve cell carcinoma, Hodgkin's Disease, Non-Hodgkin's Lymphoma, multiple myeloma, neuroblastoma, breast cancer, triple-negative breast cancer, ovarian cancer, lung cancer, primary thrombocytosis, primary macroglobulinemia, small cell lung cancer, ovarian serous cystadenocarcinoma, primary brain tumors, stomach cancer, colon cancer, malignant pancreatic insulinoma, malignant carcinoid, premalignant skin lesions, testicular cancer, prostate neuroendocrine cancer, testicular germ cell tumor, lymphomas, thyroid carcinoma, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungual melanoma, and superficial spreading melanoma.

In some embodiments, the mesothelioma is sarcomatoid mesotheliomas(S) or epithelioid mesotheliomas (E). In some embodiments, the cancer is a sarcoma. In some embodiments, the sarcoma is angiosarcoma, dermatofibrosarcoma, epithelioid sarcoma, Ewing's sarcoma, fibrosarcoma, gastrointestinal stromal tumors (GIST), Kaposi sarcoma, leiomyosarcoma, liposarcomas, dedifferentiated liposarcoma, pleomorphic myxoid liposarcoma, malignant fibrous histiocytoma, undifferentiated pleomorphic sarcoma, neurofibrosarcoma, rhabdomyosarcoma (RMS), embryonal rhabdomyosarcoma, alveolar rhabdomyosarcoma, synovial sarcoma, alveolar soft part sarcoma, chondrosarcoma, desmoid sarcoma, malignant peripheral nerve sheath tumor (MPNST), uterine carcinosarcoma, myxofibrosarcoma, undifferentiated sarcoma NOS, INI-deficient soft tissue sarcoma NOS, malignant peripheral nerve sheath tumor, desmoplastic small round cell tumor, spindle cell/sclerosing rhabdomyosarcoma, undifferentiated spindle cell sarcoma, atypical teratoid/rhabdoid tumor, fibromatosis, inflammatory myofibroblastic tumor, infantile fibrosarcoma, sclerosing epithelioid fibrosarcoma, myofibromatosis, undifferentiated hepatic sarcoma, endometrial stromal sarcoma, lipoblastomatosis, sarcomatoid disease, melanocytic clear cell sarcoma (such as soft tissue melanoma), dermal pleomorphic sarcoma, or osteosarcoma.

In some embodiments, the cancer is a sarcomatoid carcinoma. In some embodiments, the sarcomatoid carcinoma is associated with the subject's skin, bone, mouth, thyroid gland, breast, stomach, small intestine, liver, pancreas, uterus, prostate, bladder, kidneys, mediastinum, or lung.

In some aspects, described herein are methods of treating cancer in a subject in need thereof. The method may comprise: (i) receiving information on the quantitative expression of SLC7A11 protein in cancer tissue, and (ii) administering to the subject an effective amount of a PRX3 inhibitor if the quantitative expression of SLC7A11 protein in cancer tissue is lower than or similar to the quantitative expression in normal or healthy tissue; or administering to the subject an effective amount of a PRX3 inhibitor and SLC7A11 inhibitor if the quantitative expression of SLC7A11 protein in cancer tissue is higher than the quantitative expression in normal or healthy tissue.

In some embodiments, the PRX3 inhibitor is thiostrepton. In some embodiments, the PRX3 inhibitor decreases the mesenchymal cell phenotype or increases the epithelial cell phenotype. In some embodiments, the SLC7A11 inhibitor is riluzole, sulfasalazine, erastin, sorafenib, or lanperisone. In some embodiments, the method further comprises administering an effective amount of a cancer chemotherapeutic agent. In some embodiments, the cancer chemotherapeutic agent is selected from the group consisting of thiotepa, cyclophosphamide, busulfan, improsulfan, piposulfan, benzodopa, carboquone, meturedopa, uredopa, altretamine, triethylenemelamine, triethylenephosphoramide, triethylenethiophosphoramide, trimethylolmelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin; neocarzinostatin chromophore, aclacinomycins, actinomycin, authramycin, azaserine, bleomycins, cactinomycin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatrexate, demecolcine, diaziquone, eflornithine, elliptinium acetate, epothilone, etoglucid, gallium nitrate, hydroxyurea, lentinan, lonidamine, maytansine, ansamitocins, mitoguazone, mitoxantrone, mopidamol, nitracrine, pentostatin, phenamet, pirarubicin, losoxantrone, podophyllinic acid, 2-ethylhydrazide, procarbazine, PSK polysaccharide complex, razoxane, rhizoxin, sizofiran, spirogermanium, tenuazonic acid, triaziquone; 2,2′,2″-trichlorotriethylamine, trichothecene, T-2 toxin, verrucarin A, roridin A, anguidine, urethane, vindesine, dacarbazine, mannomustine, mitobronitol, mitolactol, pipobroman, gacytosine, arabinoside, cyclophosphamide, paclitaxel, docetaxel, chlorambucil, gemcitabine, 6-thioguanine, mercaptopurine, methotrexate, cisplatin, oxaliplatin, carboplatin, vinblastine, platinum, etoposide, ifosfamide, vincristine, vinorelbine, novantrone, teniposide, edatrexate, daunomycin, aminopterin, xeloda, ibandronate, irinotecan, RFS 2000, difluoromethylornithine, retinoic acid, and capecitabine.

In some embodiments, the cancer is selected from the group consisting of sarcoma, sarcomatoid carcinoma, thymoma, glioblastoma multiforme, adrenocortical cancer, uterine carcinosarcoma, uterine corpus endometrioid carcinoma, kidney clear cell carcinoma, mesothelioma, brain lower grade glioma, pheochromocytoma and paraganglioma, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, KRAS-mutant melanoma, renal cell carcinoma of kidney, kidney papillary cell carcinoma, reserve cell carcinoma, Hodgkin's Disease, Non-Hodgkin's Lymphoma, multiple myeloma, neuroblastoma, breast cancer, triple-negative breast cancer, ovarian cancer, lung cancer, primary thrombocytosis, primary macroglobulinemia, small cell lung cancer, ovarian serous cystadenocarcinoma, primary brain tumors, stomach cancer, colon cancer, malignant pancreatic insulinoma, malignant carcinoid, premalignant skin lesions, testicular cancer, prostate neuroendocrine cancer, testicular germ cell tumor, lymphomas, thyroid carcinoma, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungual melanoma, and superficial spreading melanoma.

In some embodiments, the mesothelioma is sarcomatoid mesotheliomas(S) or epithelioid mesotheliomas (E). In some embodiments, the cancer is a sarcoma. In some embodiments, the sarcoma is angiosarcoma, dermatofibrosarcoma, epithelioid sarcoma, Ewing's sarcoma, fibrosarcoma, gastrointestinal stromal tumors (GIST), Kaposi sarcoma, leiomyosarcoma, liposarcomas, dedifferentiated liposarcoma, pleomorphic myxoid liposarcoma, malignant fibrous histiocytoma, undifferentiated pleomorphic sarcoma, neurofibrosarcoma, rhabdomyosarcoma (RMS), embryonal rhabdomyosarcoma, alveolar rhabdomyosarcoma, synovial sarcoma, alveolar soft part sarcoma, chondrosarcoma, desmoid sarcoma, malignant peripheral nerve sheath tumor (MPNST), uterine carcinosarcoma, myxofibrosarcoma, undifferentiated sarcoma NOS, INI-deficient soft tissue sarcoma NOS, malignant peripheral nerve sheath tumor, desmoplastic small round cell tumor, spindle cell/sclerosing rhabdomyosarcoma, undifferentiated spindle cell sarcoma, atypical teratoid/rhabdoid tumor, fibromatosis, inflammatory myofibroblastic tumor, infantile fibrosarcoma, sclerosing epithelioid fibrosarcoma, myofibromatosis, undifferentiated hepatic sarcoma, endometrial stromal sarcoma, lipoblastomatosis, sarcomatoid disease, melanocytic clear cell sarcoma (such as soft tissue melanoma), dermal pleomorphic sarcoma, or osteosarcoma.

In some embodiments, the cancer is a sarcomatoid carcinoma. In some embodiments, the sarcomatoid carcinoma is associated with the subject's skin, bone, mouth, thyroid gland, breast, stomach, small intestine, liver, pancreas, uterus, prostate, bladder, kidneys, mediastinum, or lung.

### BRIEF DESCRIPTION OF THE DRAWINGS

FIGS. 1A-1F show development and characterization of thiostrepton (TS)-resistant cells. FIG. 1A shows TS_tolerant cells with less viability when treated with TS compared to H-meso parent cells. FIG. 1B shows TS_tolerant cells with significant reduction in cell proliferation when treated with TS compared to H-meso parent cells. FIG. 1C shows TS_tolerant cells with morphological changes different from the H-meso parent cells. FIG. 1D shows changes in gene expression in response to TS exposure. FIG. 1E shows cell viability assay of HM, HM-TSR (TS-resistant), and HM-TSR cells removed from TS pressure for 20 passages. Partial resistance of HM-TSR cells is reversible when pressure is removed. FIG. 1F shows that HM-TSR cells removed from TS pressure (HM-TSR (-TS)) retain a proliferation defect (n=4).

FIGS. 2A-2F show E to S transition pathway. FIG. 2A shows that S regions exhibited higher cell density (Dₛ<Dₑ), associated with upregulated hypoxia, ferroptosis, angiogenesis and glycolysis (S=Sarcomatoid, E=epithelioid). FIG. 2B shows an E to S transition pathway uncovered by geospatial analysis. FIG. 2C shows that S regions upregulated transcription of TGF-beta, and epithelial mesenchymal transition (EMT) associated genes. EMT, TGF-β, and Hypoxia pathways are enriched in sarcomatoid regions. FIG. 2D shows GSEA analysis comparing E and S transcriptomes. S regions upregulated hypoxia, glycolysis, angiogenesis and ferroptosis transcription consistent with increased oxidative stress. FIG. 2E shows that hypoxia, TGFβ and EMT signature enrichment were highly correlated in an orthogonal cohort of 150 mesotheliomas. FIG. 2F shows that spatial multiregional sampling of adjacent, intra-tumor E (7) vs S (12) regions from 7 patients with bi-phasic mesothelioma, was followed by transcriptome analysis of tumor regions.

FIG. 3 shows E to S genomic evolution.

FIGS. 4A-4K show E to S transition requires Mitochondrial peroxiredoxin 3 (PRX3). FIG. 4A shows PRX3 inhibitor RSO-021 (Thiostrepton, TS), downregulated Transcriptional enhanced associate domain (TEAD) target genes, TGF-β and EMT associated genes including CDH2 and upregulated mesothelin in biphasic mesothelioma cells. FIG. 4B shows GSEA analysis comparing control to TS treated cell lines. EMT, hypoxia and glycolysis associated transcription were reduced in RSO-021 treated cells. FIG. 4C shows RNA-Seq showing canonical EMT genes are downregulated by TS and in TSR cells. TGF-β, CDH2 (n-cadherin), and EGFR were downregulated in RSO-021 treated cells. FIG. 4D shows that previously described EMT genes associated with mesothelioma are modulated by TS and in TSR cells. Mesothelin (MSLN) and VISTA were upregulated in RSO-021 treated cells. FIG. 4E shows the TEAD pathway. FIG. 4F shows that MAPK 13 (p38) expression is increased in TS treated cells. FIG. 4G shows that TGFb1 and TGFb2 are downregulated in TS treated cells. FIG. 4H shows that VGLL4 inhibits TEAD transcription. FIG. 4I shows additional TEAD targets downregulated by TS treatment: EGFR, DKK1, WNT5B, SLC7A5, and WNT5A (FIG. 4I). FIG. 4J shows that RNA-Seq was conducted on human biphasic mesothelioma cell lines treated with TS for 18hrs and compared to untreated cells (n=3 biological replicates. FIG. 4K shows protein expression of N-cadherin and MSLN in HM-TSR cells and HM-TSR cells that have been removed from TS pressure for 20 passages. Changes to protein expression are maintained.

FIGS. 5A-5B show epithelial to mesenchymal phenotype. FIG. 5A that shows mesothelin (MSLN) is exclusively expressed in epithelioid MM. TS resistant cells increase MSLN expression, indicating a switch to an epithelioid phenotype. FIG. 5B shows that EMT is a primary driver in the Epithelioid to sarcomatoid transition.

FIGS. 6A-6D show that epithelial mesenchymal transition genes are downregulated in patient derived explants (PDEs) treated with TS. FIG. 6A shows schematic of experiments conducted with PDEs. FIG. 6B shows that gene set enrichment analysis (GSEA) method for Hallmark collection revealed that gene set related to EMT is enriched in untreated explants compared to those treated with 10 μM of TS. FIG. 6C shows enrichment plot for Hallmark EMT gene set. FIG. 6D shows that comparison of values for Hallmark EMT gene set for untreated PDEs and those treated with 10 μM of TS obtained by gene set variation analysis (GSVA) confirmed that genes associated with EMT are enriched in control samples.

FIGS. 7A-7B show administration of RSO-021 and effect on cytokine levels. FIG. 7A shows that RSO-021 is administered once-a-week via an indwelling intrapleural catheter (IPC) until disease progression, unacceptable toxicity, withdrawal of consent or study termination. Prior to each dose patients will have pleural effusion drained to dryness per standard of care. After each administration, the IPC is secured until next RSO-021 dosing time point. FIG. 7B shows changes in specific cytokine levels at C1D1 (Pre-RSO-021), and post RSO-021 (C1D2-C5D1). Absolute values as determined following ELISA are shown from 7 patients enrolled on the trial.

FIGS. 8A-8D show that TS activity is independent of BAP1 expression. FIG. 8A show BAP1 protein expression in immortalized mesothelial (LP9), pleural Meso (HM, MI), and peritoneal Meso (Pet-Gar) cell lines. TS treatment at 4 μM does not alter BAP1 protein expression. FIG. 8B show EC50 values for TS in normal and MM cell lines (*p<0.05, **p<0.01, ***p<0.001, ****p<0.0001, n=4 biological replicates) FIG. 8C show protein western blots of BAP1 and GAPDH in HM scramble control (SCR) and BAP1 siRNA expressing (siBAP 1) cells. FIG. 8D show cell viability curves of cells treated with TS 48 hours (n=3).

FIGS. 9A-9C show that TS and erastin have synergistic activity in Meso cells. FIG. 9A shows that synergy distribution plot and Zip Synergy Score (ZSS) for Erastin tested in combination with TS. ZSS>10 indicates synergy between the two compounds. FIG. 9B shows sensitivity of HM cells to TS, Erastin, or TS+erastin (N=2). FIG. 9C shows cell viability curves of HM cells treated with TS in media+/−cystine supplementation (N=2).

FIGS. 10A-10D show that erastin potentiates PRX3 crosslinking and ROS generation by TS. FIG. 10A show Western blot from HM cells treated with TS (1 μM), erastin (ER, 2.5 μM) or TS+ER for 24 hours. Samples were run under reducing conditions by SDS-PAGE. FIG. 10B show quantification of PRX3 dimer (TS-crosslink) to PRX3 monomer (unmodified PRX3) from protein western blots as in A (n=2 biological replicates). FIG. 10C show ROS levels in cells treated with TS, ER, or TS+ER for 24 hours (n=2 biological replicates, 3 technical replicates). FIG. 10D show protein western blot of cells cultured in media+/−cystine and treated with TS or ER. PRX3 crosslinking by TS is increased in cells cultured in cystine free (−) media.

FIGS. 11A-11C show that TS synergy with ER is independent of ferrostatin sensitive MOA. FIG. 11A shows cell viability curves of cells treated with indicated compounds for 48 hours. Ferrostatin (Fer-1) blocks ER cytotoxicity but not TS cytotoxicity FIG. 11BA shows cell viability curves of cells treated with indicated compounds for 48 hours. Fer-1 does not block TS/ER cytotoxic synergy (green vs. purple lines). FIG. 11C shows protein western blot of PRX3 crosslinking by TS, ER, and TS+ER in cells treated+/−Fer-1. Fer-1 does not alter PRX3 crosslinking by TS or TS+ER.

FIGS. 12A-12C show that knockdown of SLC7A11 potentiates TS activity. FIG. 12A shows SLC7A11 expression measured by RT-qPCR in HM cells at 48 hrs post transfection with indicated siRNA (Ctrl nontransfected, scramble 1 (SCR 1), scramble 2 (SCR 2) and SLC7A11 siRNA) n=3 biological replicates. FIG. 12B shows cell viability assay of HM cell line with indicated permutations treated with TS, n=2 biological replicates. FIG. 12C shows Western blot of PRX3 in the HM cell lines with indicated permutations. PRX3 crosslinking is increased in the SLC7A11 KD samples.

FIGS. 13A-13B show TS-resistant cells over-express SLC7A11 and are re-sensitized to TS by Erastin (ER). FIG. 13A shows Western blot of SLC7A11 levels in LP9, HM and HM-TSR cells. FIG. 13B shows cell viability assay of HM and HM-TSR cells treated with TS, ER or TS+ER for 48 hours. The shift in sensitivity of the HM-TSR-TS line to the HM-TSR-TS/ER line.

FIGS. 14A-14B show that SLC7A11 is enriched in patient derived explants (PDEs) that are non responders to TS. FIG. 14A shows DESeq2 analysis of responders (R) and non-responders (NR) group showed SLC7A11 upregulation in NR. Assignment to R or NR was based on cPARP staining results in treated explants in comparison to untreated controls. FIG. 14B shows that this phenomenon was confirmed in Mann-Whitney test conducted on normalized transcript level values (fpkm) for R and NR groups.

### DETAILED DESCRIPTION OF THE INVENTION

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N. Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N. Y. (1999); and Gilbert et al., “Developmental Biology, 6th ed.”, Sinauer Associates, Inc., Sunderland, MA (2000).

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given ligand) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

“Immunotherapy” is treatment that uses a subject's immune system to treat cancer and includes, for example, checkpoint inhibitors, cancer vaccines, cytokines, cell therapy, CAR-T cells, and dendritic cell therapy.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., when at least 5% of drug product is detectable systemically with industry acceptable methodology, or when the two agents are simultaneously effective in the patient, which may include synergistic effects of the two agents). For example, the different therapeutic compounds can be administered either in the same formulation or in separate formulations, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

### Pharmaceutical Composition

The compositions and methods described herein may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound described herein and a pharmaceutically acceptable carrier. Pharmaceutically acceptable carriers are well known in the art and include, as a non-limiting example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound described herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound described herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, com oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound described herein, with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

Formulations described herein suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound described herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound described herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods described herein, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors that influence the effective amount may include, but are not limited to, the severity of the patient's condition, the disorder being treated, the stability of the compound, and, if desired, another type of therapeutic agent being administered with the compound described herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods described herein will be that amount of the compound that is the lowest dose effective to produce a therapeutic effect. Such an effective dose will generally depend upon the factors described above. If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds described herein may be used alone or conjointly administered with another type of therapeutic agent.

The present disclosure includes the use of pharmaceutically acceptable salts of compounds described herein in the compositions and methods described herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn or other metal salts. In certain embodiments, contemplated salts include, but are not limited to, 1-hydroxy-2-naphthoic acid, 2,2-dichloroacetic acid, 2-hydroxy ethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, 1-pyroglutamic acid, salicylic acid, sebacic acid, stearic acid, succinic acid, sulfuric acid, L-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BEIT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Method of Treatment

RSO-021 is a PRX3 inhibitor that was investigated in a first-in-human phase ½ clinical trial, recruiting patients with malignant pleural effusion (MPE) arising from mesothelioma or other solid tumors. Mesothelioma cells rely on buffering of mitochondrial oxidative stress through the expression and activity of mitochondrial PRX3, which is covalently inactivated by RSO-021. Inactivation of PRX3 by RSO-021 leads to increased oxidative stress and tumor cell death. RSO-021 may be administered weekly via an intrapleural catheter. Cytokine levels in pleural effusion drainages were measured by ELISA assays. Tumor explants were grown following surgical resection of mesothelioma tumors. RNA sequencing of MM cell lines was conducted. Examination of pleural effusion pre-, and post RSO-021 treatment has revealed significant changes to cytokine levels including Resistin, IL-8 and IL-6. RNA sequencing of cells derived from pre-and post-treated MPE aims to identify RSO-021 induced transcriptional changes and their association with clinical activity. Furthermore, a co-clinical trial involving live mesothelioma explants has been completed, enabling parallel assessment of RSO-021 efficacy, and immune modulation. A geospatial transcriptomic gradient was identified between adjacent epithelioid and sarcomatoid regions in biphasic mesotheliomas involving hypoxia tolerance, TGF-b, mitochondrial oxidative phosphorylation, and epithelial to mesenchymal transition (EMT). Treatment of human biphasic mesothelioma cells with RSO-021 downregulated TEAD target genes, TGF-b and EMT associated genes including CDH2, indicating that PRX3 inhibition might reverse or slow the epithelioid to sarcomatoid transition. These studies reveal tumor cell and tumor microenvironment changes elicited by the PRX3 inhibitor RSO-021.

Mesothelioma, a lethal cancer associated with asbestos exposure, lacks effective drug therapy particularly in the relapsed treatment setting. Epithelial-to-mesenchymal transition (EMT) confers an aggressive sarcomatoid phenotype capable of invasion, metastasis, and drug resistance. Pharmacological targeting of EMT could favorably alter the progression and treatment of mesothelioma. Mesothelioma cells rely on buffering of mitochondrial oxidative stress through the expression and activity of mitochondrial peroxiredoxin 3 (PRX3), which is covalently inactivated by the inhibitor RSO-021 (Thiostrepton, TS). To better understand the pharmacodynamics of TS, transcriptomic profiles of drug sensitive and resistant biphasic mesothelioma cell lines were generated. Differential analysis revealed that TS induced upregulation of ROS response and apoptosis signaling, while downregulating TGF-β and EMT markers. Additionally, we observed altered expression of the mesothelioma specific EMT genes Col5A, mesothelin, and VISTA. Resistance to TS conferred significant proliferative defects and was reversible upon TS removal, however, the downregulation of EMT genes was conserved, suggesting an irreversible mesenchymal-to-epithelial transition. Lastly, downregulation of EMT by TS was determined in a cohort of TS treated mesothelioma surgical explants. In summary, PRX3 is a clinically actionable drug target, inhibition of which correlates with EMT modulation.

Spatial transformation in mesothelioma involves a hypoxia-TGFb-EMT axis that is reversible via PRX3 inhibition. Sarcomatoid malignant pleural mesotheliomas(S) exhibit an aggressive, drug-resistant phenotype with invasive properties compared with epithelioid mesotheliomas (E). Understanding the intra-tumour processes that underpin E to S transition in biphasic mesotheliomas could highlight opportunities to block or reverse this transformation. The data described herein show that sarcomatoid transformation exhibits adaption to hypoxia via a TGF-b/TEAD/EMT axis, which may be reversed by PRX3 inhibition.

Microenvironmental cell density and hypoxia may be a key driver of E to S transition with glycolytic, angiogenic and redox adaptions. PRX3 appears to be essential for stabilizing the EMT phenotype. The data described herein show that sarcomatoid transformation involves a hypoxia/TGF-b/TEAD/EMT axis which may be reversed by PRX3 inhibition. EMT may be a selection pressure for Hippo inactivation which occurs as a later evolutionary somatic event.

In certain aspects, described herein is a method of treating cancer in a subject in need thereof comprising administering to the subject an effective amount of a PRX3 inhibitor.

In some aspects, described herein are methods of treating cancer in a subject in need thereof comprising determining or receiving information on the quantitative expression of a biomarker associated with epithelial-mesenchymal transition (EMT), wherein the biomarker is characteristic for a mesenchymal cell phenotype or for an epithelial cell phenotype. The method further may comprise administering to the subject an effective amount of a PRX3 inhibitor if the biomarker characteristic for a mesenchymal cell phenotype is increased or if the biomarker characteristic for an epithelial cell phenotype is decreased.

In some aspects, described herein are methods of treating cancer in a subject in need thereof. In some embodiments, the subject comprises administering to the subject an effective amount of a PRX3 inhibitor and a SLC7A11 inhibitor. In some embodiments, the PRX3 inhibitor is thiostrepton.

In some aspects, described herein are methods of treating cancer in a subject in need thereof. The method may comprise: (i) receiving information on the quantitative expression of SLC7A11 protein in cancer tissue, and (ii) administering to the subject an effective amount of a PRX3 inhibitor if the quantitative expression of SLC7A11 protein in cancer tissue is lower than or similar to the quantitative expression in normal or healthy tissue; or administering to the subject an effective amount of a PRX3 inhibitor and SLC7A11 inhibitor if the quantitative expression of SLC7A11 protein in cancer tissue is higher than the quantitative expression in normal or healthy tissue.

Epithelial-mesenchymal transitions (EMTs), the acquisition of mesenchymal features from epithelial cells, occur during some biological processes and are classified into three types: the first type occurs during embryonic development, the second type is associated with adult tissue regeneration, and the third type occurs in cancer progression. EMT occurring during embryonic development in gastrulation, renal development, and the origin and fate of the neural crest is a highly regulated process, while EMT occurring during tumor progression is highly deregulated. EMT allows the solid tumors to become more malignant, increasing their invasiveness and metastatic activity. Secondary tumors frequently maintain the typical histologic characteristics of the primary tumor. These histologic features connecting the secondary metastatic tumors to the primary is due to a process called mesenchymal-epithelial transition (MET). MET has been demonstrated in different mesenchymal tumors and is the expression of the reversibility of EMT. EMT modulation could constitute an approach to avoid metastasis. Some of the targeted small molecules utilized as antiproliferative agents have revealed to inhibit EMT initiation or maintenance because EMT is regulated through signaling pathways for which these molecules have been designed.

Peroxiredoxin-3 (PRX3) is a mitochondrial antioxidant protein and member of the Prx family that can scavenge H₂O₂ in cooperation with thiol and peroxynitrite. In mammals, 6 distinct Prx family members have been identified (Prx-1 through -6). Among the Prxs, Prx-3 is unique because it is localized specifically within the mitochondria. Furthermore, in vivo transfer of the Prx-3 gene protected neurons against cell death induced by oxidative stress.

RSO-021 is a PRX3 inhibitor that inactivates PRX3 through direct covalent adduction of active site cysteine residues, in turn, inducing oxidative stress to levels incompatible with tumor cell survival. Preclinical studies of RSO-021 show that it selectively inhibits mitochondrial PRX3 in transformed cells and is well-tolerated in animal studies.

Thiostrepton is a cyclic oligopeptide antibiotic that is also known by other names such as Bryamycin, Thiactin, alaninamide, HR4S203Y18, etc. Recent studies have shown that thiostrepton also has promising anticancer activity. Mitochondrial ROS levels and PRX3 expression correlates with sensitivity to thiostrepton.

### EXAMPLES

The invention now being generally described, it will be more readily understood by reference to the following examples which are included merely for purposes of illustration of certain aspects and embodiments of the present invention, and are not intended to limit the invention.

### Example 1: Experimental Procedures

Described herein are methods to evaluate changes in gene expression in malignant mesothelioma cells (H-meso cell line) treated with thiostrepton (HM-TS) for 16 hrs and compare to HM-TSR (TS_tolerant cells) grown in the presence of 4 mM TS for several weeks. Biological triplicates were generated, RNA extracted, and RNA integrity evaluated. Cell lysates and cDNA were evaluated for known gene changes by RT-qPCR prior to library builds. Libraries were generated using the SMARTer® Stranded RNA-Seq Kit HT from Takara, pooled and analyzed on the Illumina HiSeq 1500 using a rapid run dual lane SE sequencing flow cell. Each library yielded an average of 15,000 mapped reads.

Spatial multiregional sampling of adjacent, intra-tumour E (7) vs S (12) regions from 7 patients with bi-phasic mesothelioma was followed by whole exome, transcriptome and 100-plex immunofluorescence analysis (employing cytokeratin-based cell segmentation). Machine learning (random forests) was employed to infer E to S evolutionary trajectories at the genomic, transcriptomic and protein levels. Biphasic human mesothelioma cells were treated with the PRX3 inhibitor RSO-021 (thiostrepton) and gene-expression changes were evaluated by RT-qPCR.

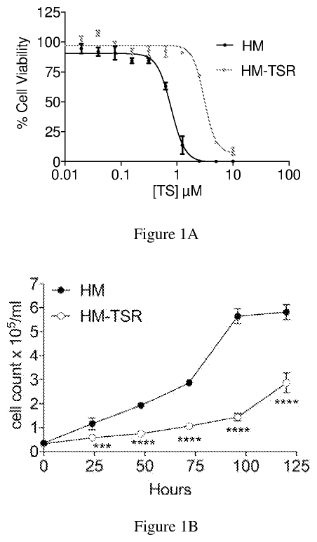

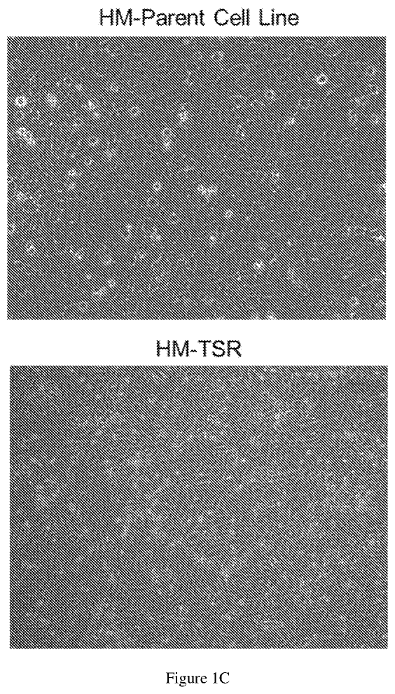

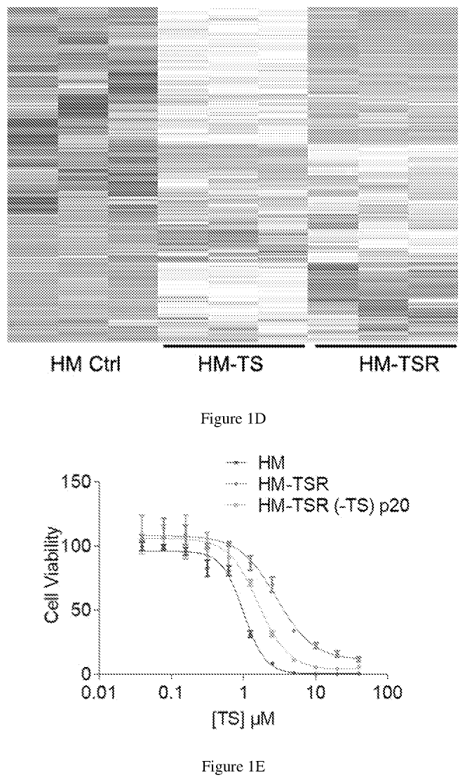

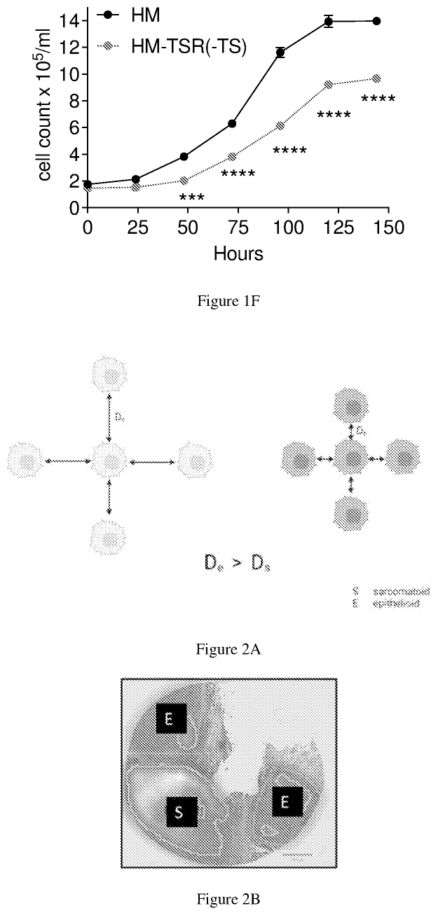

TS-resistance slows cell proliferation and is partially reversible. To generate TS-resistant cells, HM cells were dosed with TS by repetitive drug treatment with a short 24-48hr recovery period. TS concentration was gradually increased by 1.5-fold until a concentration was reached that no longer provided viable cells. Development of a tolerant cell line at 5 μM occurred after 8 months. FIG. 1E shows cell viability assay of HM, HM-TSR (TS-resistant), and HM-TSR cells removed from TS pressure for 20 passages. Partial resistance of HM-TSR cells is reversible when pressure is removed. FIG. 1B shows cell growth analysis of HM and HM-TSR cells over 125 hours (n=4). FIG. 1F shows that HM-TSR cells removed from TS pressure (HM-TSR (-TS)) retain a proliferation defect (n=4).

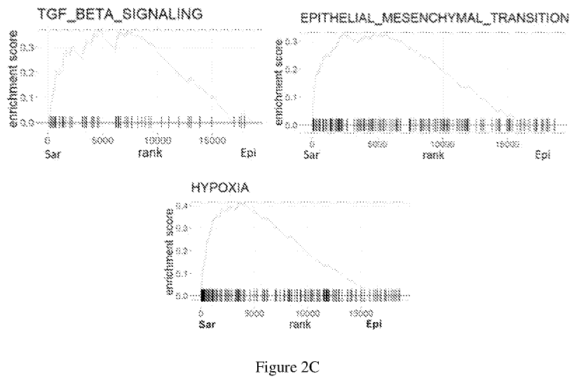

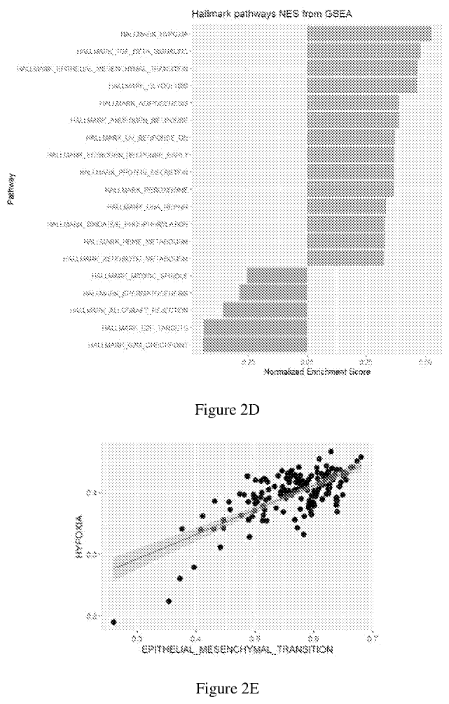

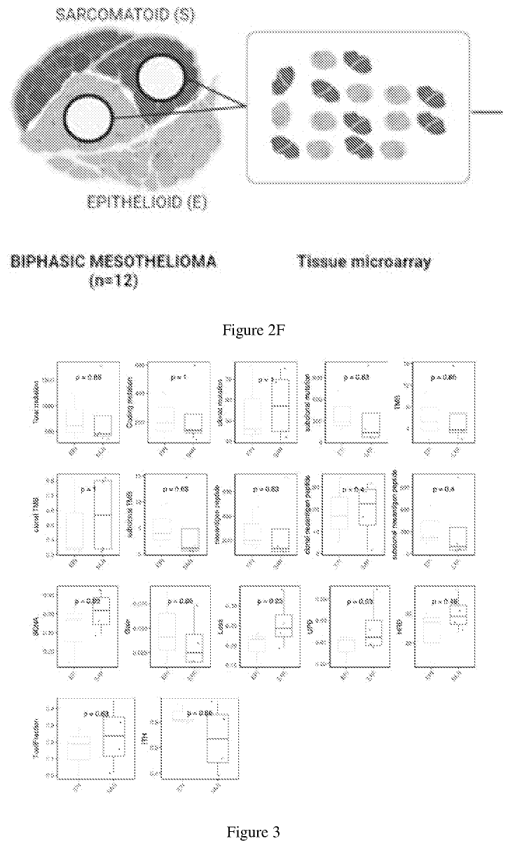

TS-tolerant cells showed less viability and significant reduction in cell proliferation when treated with TS compared to H-meso parent cells (FIGS. 1A-1B). TS_tolerant cells also showed morphological changes that are different from the H-meso parent cells (FIG. 1C). Intra-tumour E to S transition was associated with increased TEAD-dependent transcription consistent with functional inactivation of the hippo pathway. S regions upregulated transcription of TGF-beta and epithelial mesenchymal transition (EMT)-associated genes but lost expression of VISTA, argininosuccinyl synthetase, and mesothelin. E and S shared the same clonal genomic architecture with respect to mutation burden, aneuploidy, non-synonymous, neoantigen burden, and homologous DNA repair deficiency. Conversely, recurrent subclonal evolution involving negative clonal selection was observed in all patients, involving a BAP1/KMT20 and BCL9L subclones in two patients respectively. S regions upregulated hypoxia, glycolysis, angiogenesis and ferroptosis transcription consistent with increased oxidative stress (FIG. 2D). Intra-tumour E to S transition was associated with increased TEAD dependent transcription consistent with functional inactivation of the hippo pathway. S regions exhibited higher cell density (Dₛ<Dₑ), associated with upregulated hypoxia, ferroptosis, angiogenesis and glycolysis (FIG. 2A). An E to S transition pathway uncovered by geospatial analysis (FIG. 2B). S regions upregulated transcription of TGF-beta, and epithelial mesenchymal transition (EMT) associated genes (FIG. 2C) but lost expression of VISTA, argininosuccinyl synthetase, and mesothelin. A reduced expression of putative drug targets VISTA, ASS1 and mesothelin (MSLN). Hypoxia, TGFβ and EMT signature enrichment were highly correlated in an orthogonal cohort of 150 mesotheliomas (FIG. 2E).

E and S shared the same clonal genomic architecture with respect to mutation burden, aneuploidy, non-synonymous, neoantigen burden, and homologous DNA repair deficiency (FIG. 3). Conversely, recurrent subclonal evolution involving negative clonal selection was observed in all patients; involving a BAP1/KMT20 and BCL9L subclones in two patients respectively. S regions upregulated hypoxia, glycolysis, angiogenesis and ferroptosis transcription consistent with increased oxidative stress.

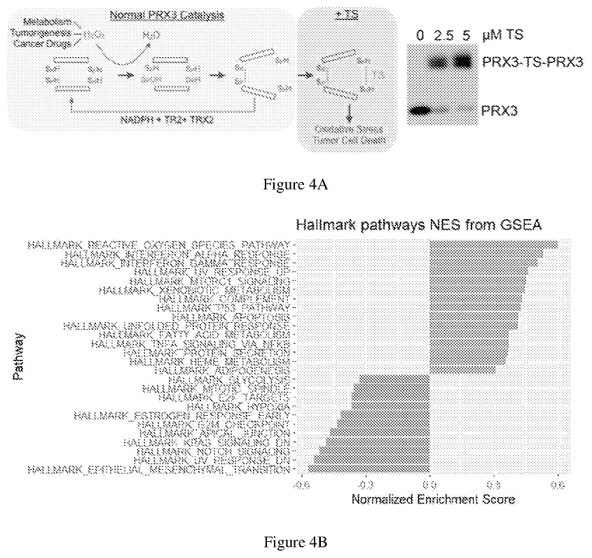

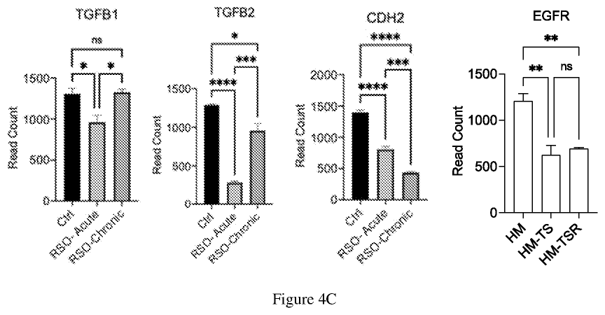

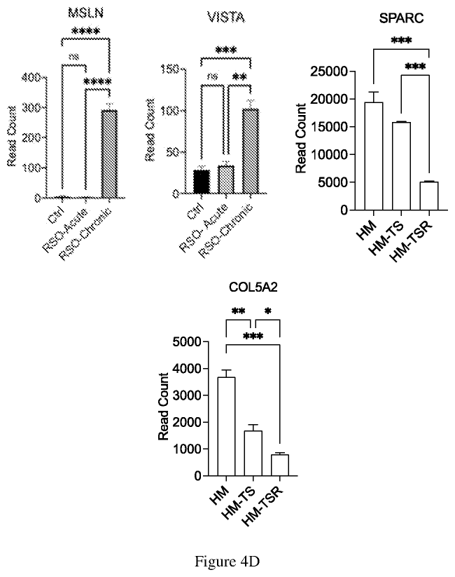

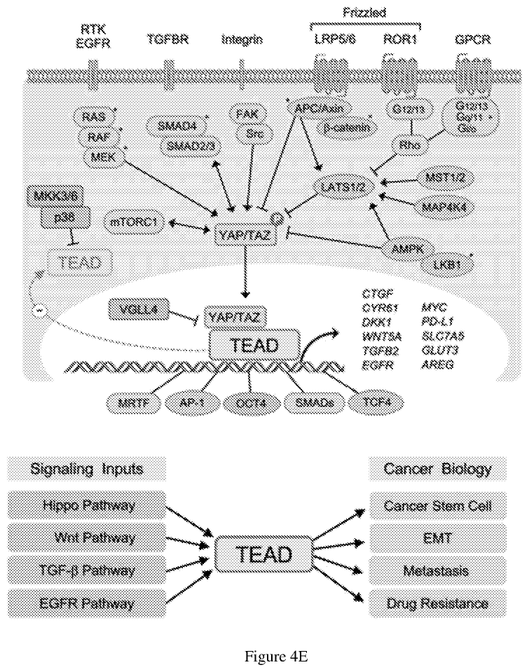

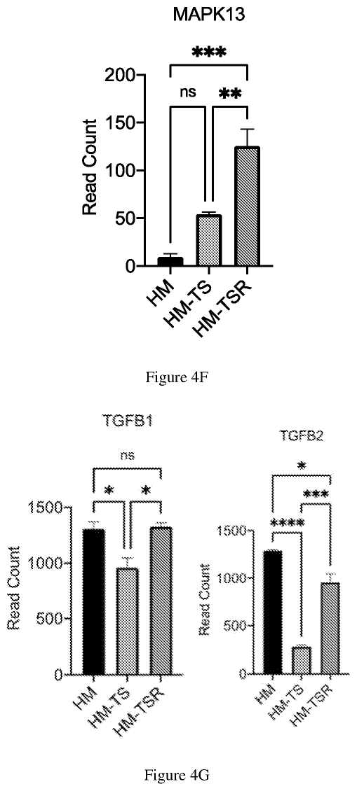

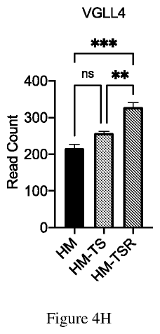

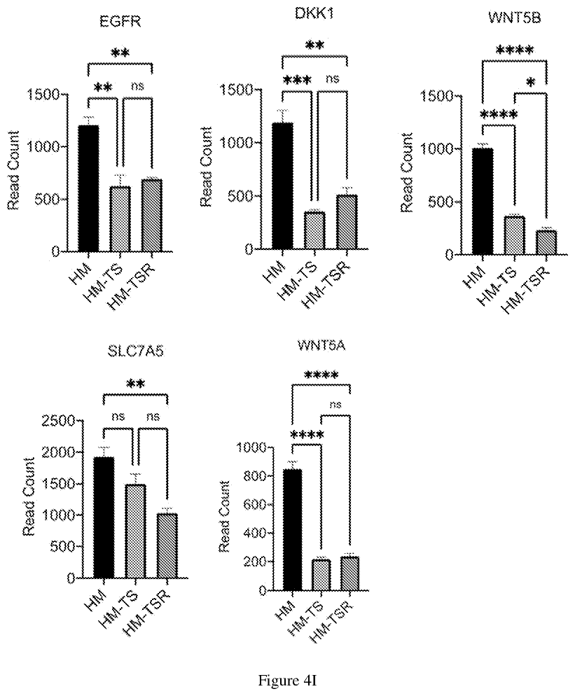

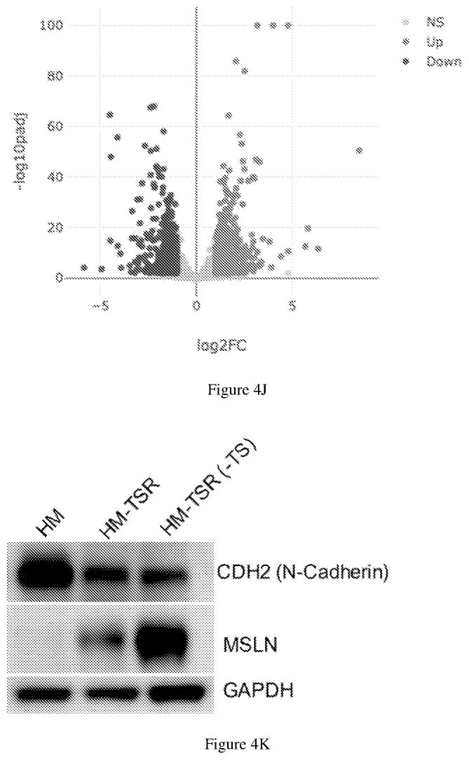

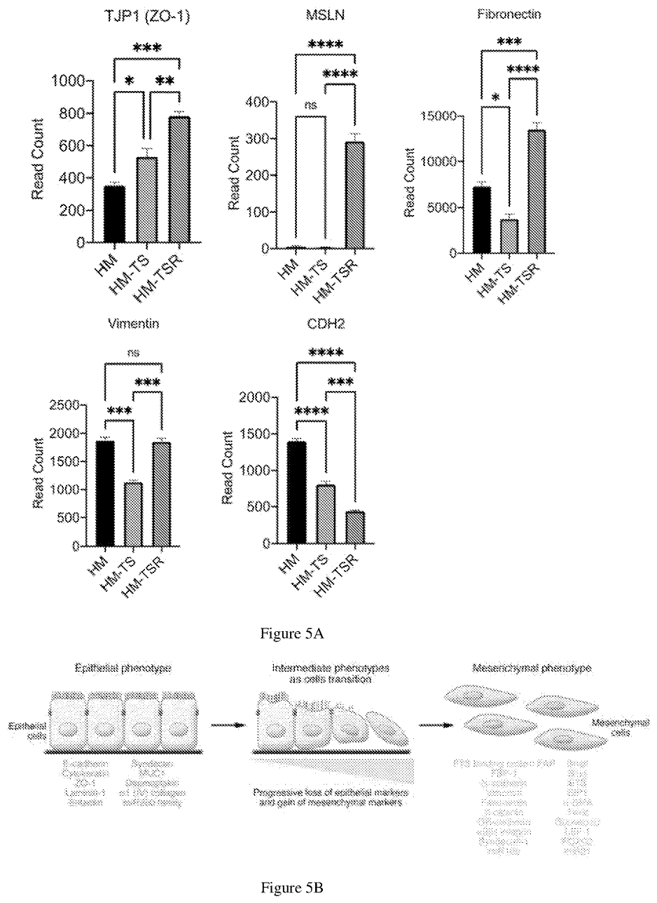

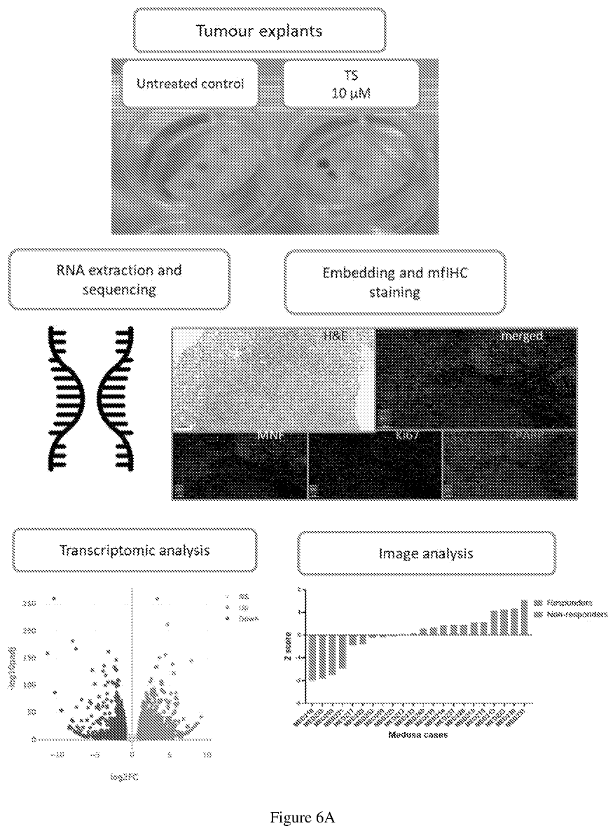

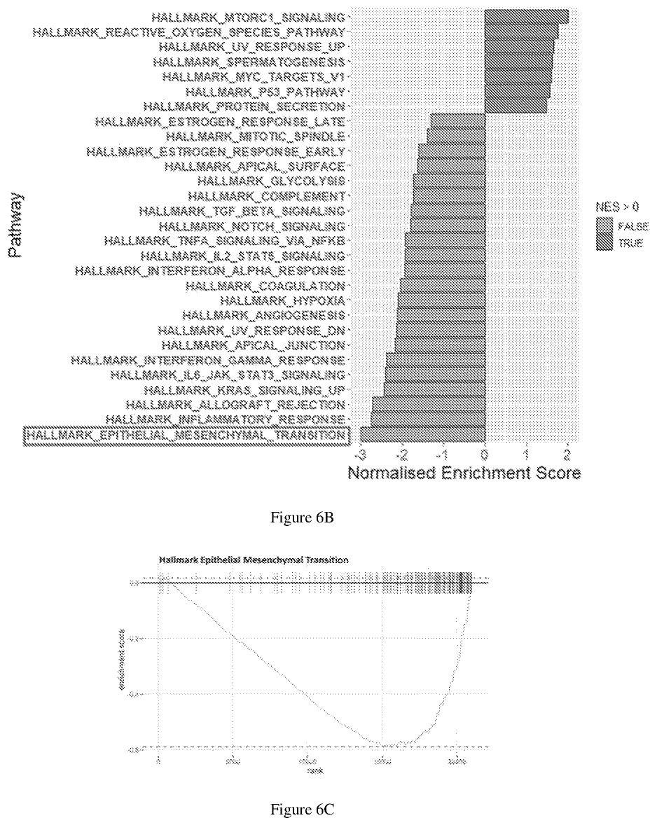

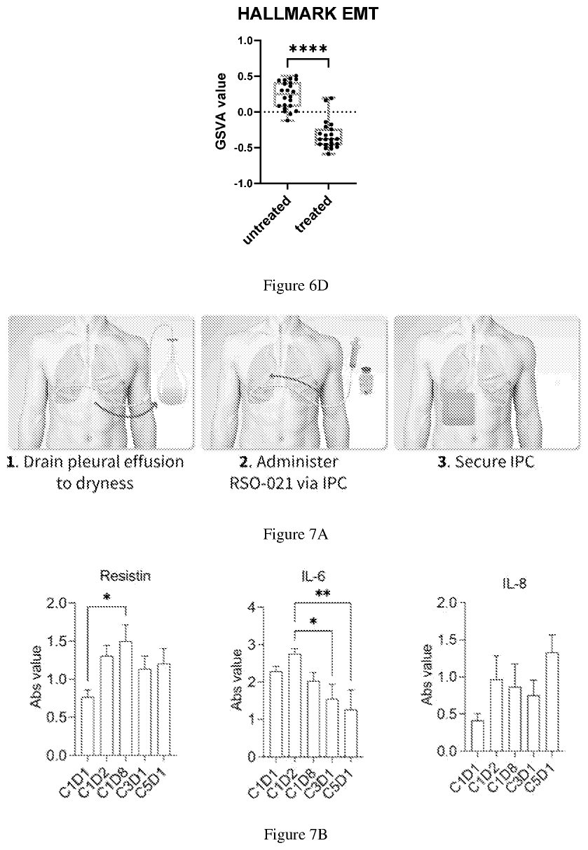

FIG. 4A shows that RSO-021 (Thiostrepton, TS) is a covalent inhibitor of mitochondrial Peroxiredoxin 3 (PRX3). PRX3 is the primary mitochondrial peroxidase required for H₂O₂ clearance from the mitochondria induced by metabolic, tumorigenic, and drug treatment inputs. During the metabolism of H₂O₂, PRX3 forms an intramolecular disulfide bond that orients the second active site for TS-dependent covalent crosslinking, inactivating the protein leading to increased oxidative stress and tumor cell death. TS-dependent modifications are visualized by reducing SDS-PAGE and western blotting where a dose-dependent irreversible increase in modified PRX3 (PRX3 -TS) is present.

E and S transition requires Mitochondrial peroxiredoxin 3 (PRX3). PRX3 inhibitor RSO-021, downregulated TEAD target genes, TGF-β and EMT associated genes including CDH2 and upregulated mesothelin in biphasic mesothelioma cells (FIG. 4A). EMT, hypoxia and glycolysis associated transcription were reduced in RSO-021 treated cells (FIG. 4B). TGF-β and CDH2 (n-cadherin) were downregulated in RSO-021 treated cells (FIG. 4C). Mesothelin (MSLN) and VISTA were upregulated in RSO-021 treated cells (FIG. 4D). FIG. 4E shows the TEAD pathway. Further, MAPK13 (p38) expression is increased in TS treated cells (FIG. 4F). p38 negatively regulates TEAD signaling while TEAD promotes EMT and tumorigenesis. TGFb1 and TGFb2, targets of TEAD transcription, are downregulated in TS treated cells (FIG. 4G). TGFb is a master regulator of EMT. VGLL4 inhibits TEAD transcription (FIG. 4H). Additional TEAD targets downregulated by TS treatment are EGFR, DKK1, WNT5B, SLC7A5, and WNT5A (FIG. 4I). Mesothelin (MSLN) is exclusively expressed in epithelioid MM (FIG. 5A). TS resistant cells increase MSLN expression, indicating a switch to an epithelioid phenotype. EMT is a primary driver in the Epithelioid to sarcomatoid transition (FIG. 5B).

### Example 2: SLC7A11 Modulates Sensitivity to the Mitochondrial Peroxiredoxin 3 Inhibitor Thiostrepton (RSO-021) via a Ferroptosis Independent Pathway

Described herein are methods to evaluate changes in gene expression in malignant mesothelioma cells (H-meso cell line) treated with thiostrepton (HM-TS) for 16 hrs and compare to HM-TSR (TS_tolerant cells) grown in the presence of 4 mM TS for several weeks. Biological triplicates were generated, RNA extracted, and RNA integrity evaluated. Cell lysates and cDNA were evaluated for known gene changes by RT-qPCR prior to library builds. Libraries were generated using the SMARTer® Stranded RNA-Seq Kit HT from Takara, pooled and analyzed on the Illumina HiSeq 1500 using a rapid run dual lane SE sequencing flow cell. Each library yielded an average of 15,000 mapped reads.

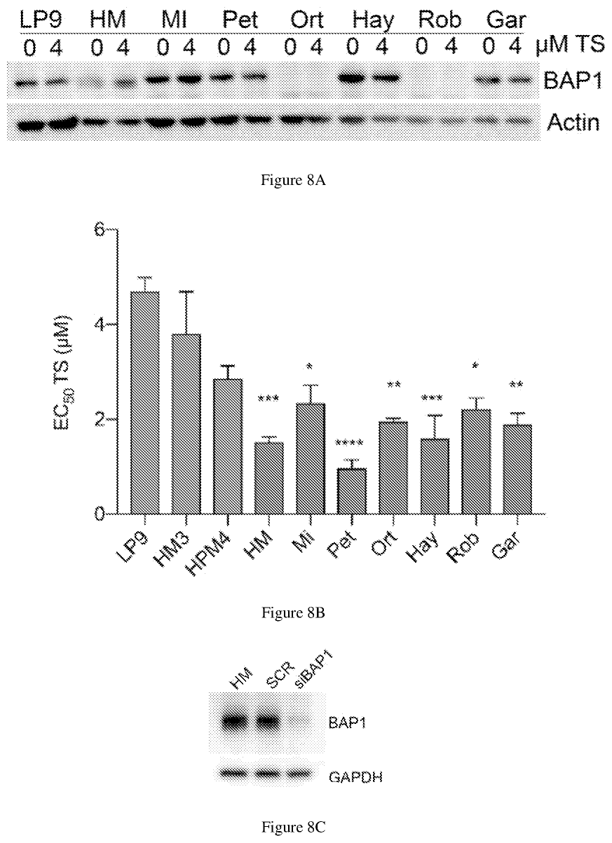

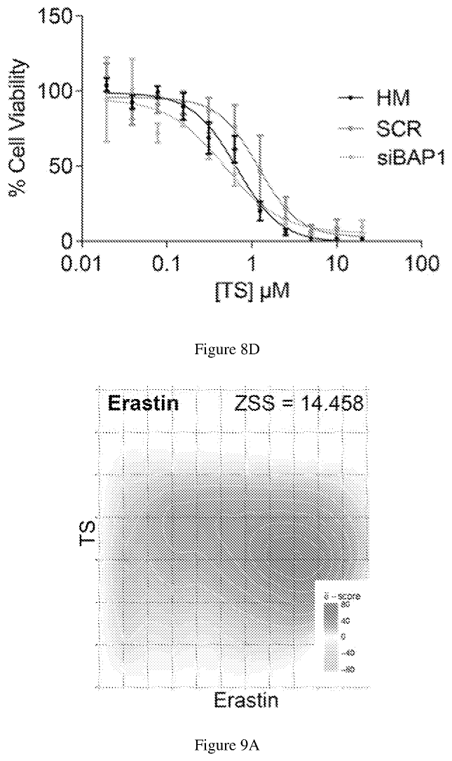

TS activity is independent of BAP1 expression. FIG. 8A show BAP1 protein expression in immortalized mesothelial (LP9), pleural Meso (HM, MI), and peritoneal Meso (Pet-Gar) cell lines. TS treatment at 4 μM does not alter BAP1 protein expression. FIG. 8B show EC50 values for TS in normal and MM cell lines (*p<0.05, **p<0.01, ***p<0.001, ****p <0.0001, n=4 biological replicates) FIG. 8C show protein western blots of BAP1 and GAPDH in HM scramble control (SCR) and BAP1 siRNA expressing (siBAP1) cells. FIG. 8D show cell viability curves of cells treated with TS 48 hours (n=3).

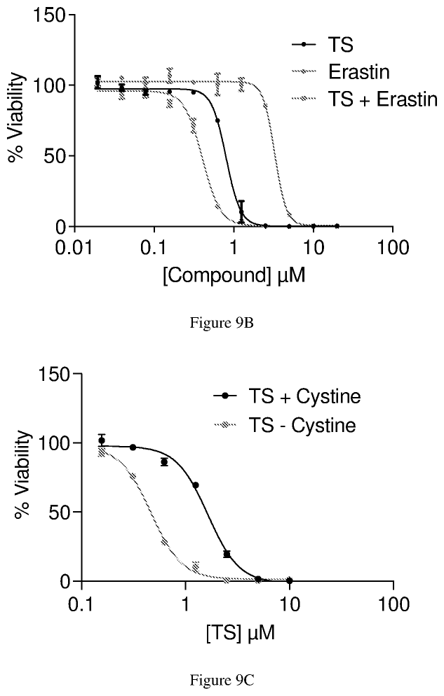

TS and erastin have synergistic activity in Meso cells. FIG. 9A shows that synergy distribution plot and Zip Synergy Score (ZSS) for Erastin tested in combination with TS. ZSS>10 indicates synergy between the two compounds. FIG. 9B shows sensitivity of HM cells to TS, Erastin, or TS+erastin (N=2). FIG. 9C shows cell viability curves of HM cells treated with TS in media+/−cystine supplementation (N=2).

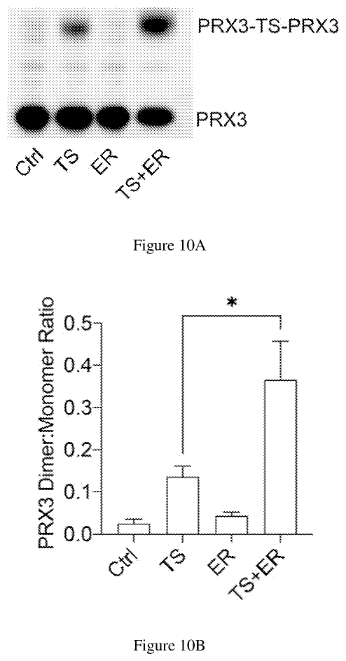

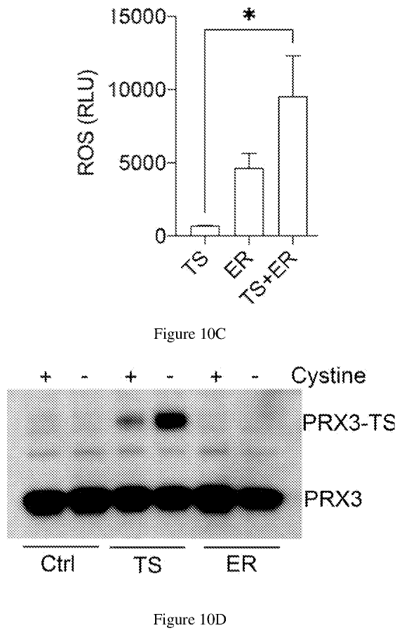

Erastin potentiates PRX3 crosslinking and ROS generation by TS. FIG. 10A show Western blot from HM cells treated with TS (1 μM), erastin (ER, 2.5 μM) or TS+ER for 24 hours. Samples were run under reducing conditions by SDS-PAGE. FIG. 10B show quantification of PRX3 dimer (TS-crosslink) to PRX3 monomer (unmodified PRX3) from protein western blots as in A (n=2 biological replicates). FIG. 10C show ROS levels in cells treated with TS, ER, or TS+ER for 24 hours (n=2 biological replicates, 3 technical replicates). FIG. 10D show protein western blot of cells cultured in media+/−cystine and treated with TS or ER. PRX3 crosslinking by TS is increased in cells cultured in cystine free (−) media.

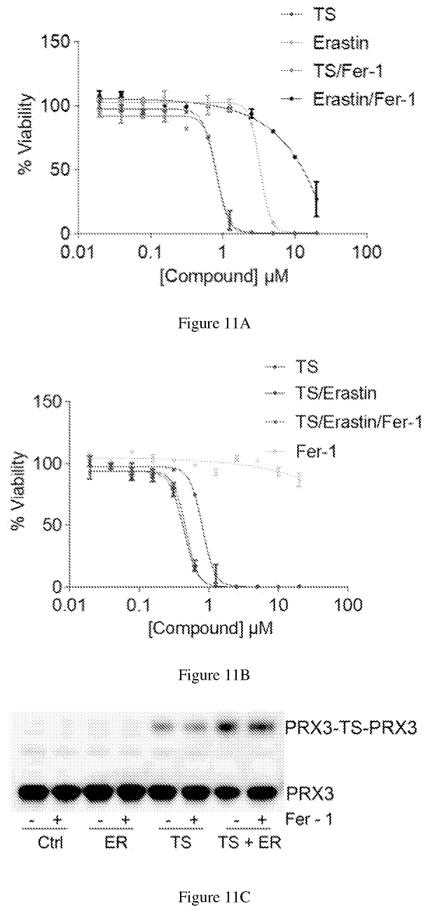

TS synergy with ER is independent of ferrostatin sensitive MOA. FIG. 11A shows cell viability curves of cells treated with indicated compounds for 48 hours. Ferrostatin (Fer-1) blocks ER cytotoxicity but not TS cytotoxicity FIG. 11BA shows cell viability curves of cells treated with indicated compounds for 48 hours. Fer-1 does not block TS/ER cytotoxic synergy (green vs. purple lines). FIG. 11C shows protein western blot of PRX3 crosslinking by TS, ER, and TS+ER in cells treated+/-Fer-1. Fer-1 does not alter PRX3 crosslinking by TS or TS+ER.

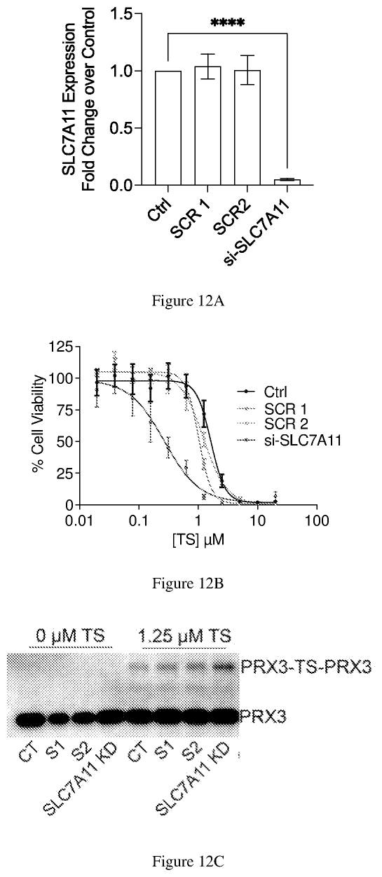

Knockdown of SLC7A11 potentiates TS activity. FIG. 12A shows SLC7A11 expression measured by RT-qPCR in HM cells at 48 hrs post transfection with indicated siRNA (Ctrl nontransfected, scramble 1 (SCR 1), scramble 2 (SCR 2) and SLC7A11 siRNA) n=3 biological replicates. FIG. 12B shows cell viability assay of HM cell line with indicated permutations treated with TS, n=2 biological replicates. FIG. 12C shows Western blot of PRX3 in the HM cell lines with indicated permutations. PRX3 crosslinking is increased in the SLC7A11 KD samples.

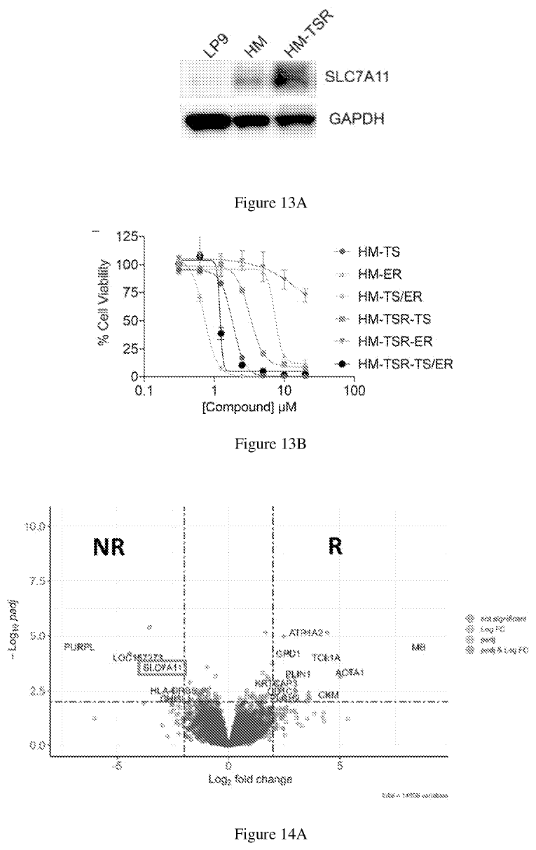

TS-resistant cells over-express SLC7A11 and are re-sensitized to TS by Erastin (ER). HM cells were dosed with TS by repetitive drug treatment with a short 24-48 hr recovery period. TS concentration was gradually increased by 1.5-fold until a concentration was reached that no longer provided viable cells. Development of a drug-tolerant cell line with tolerance at 5 M after 8 months. FIG. 13A shows Western blot of SLC7A 11 levels in LP9, HM and HM-TSR cells. FIG. 13B shows cell viability assay of HM and HM-TSR cells treated with TS, ER or TS+ER for 48 hours. The shift in sensitivity of the HM-TSR-TS line to the HM-TSR-TS/ER line.

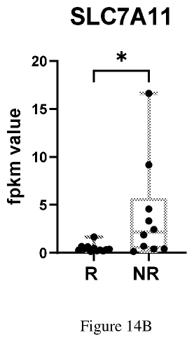

SLC7A11 is enriched in patient derived explants (PDEs) that are non responders to TS. FIG. 14A shows DESeq2 analysis of R and NR group showed SLC7A11 upregulation in NR. FIG. 14B shows that this phenomenon was confirmed in Mann-Whitney test conducted on normalized transcript level values (fpkm) for R and NR groups.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating cancer in a subject in need thereof comprising administering to the subject an effective amount of a PRX3 inhibitor, wherein the cancer is selected from the group consisting of sarcomatoid mesotheliomas(S), epithelioid mesotheliomas (E), sarcoma, sarcomatoid carcinoma, thymoma, adrenocortical cancer, uterine carcinosarcoma, uterine corpus endometrioid carcinoma, kidney clear cell carcinoma, brain lower grade glioma, pheochromocytoma and paraganglioma, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, KRAS-mutant melanoma, renal cell carcinoma of kidney, kidney papillary cell carcinoma, reserve cell carcinoma, triple-negative breast cancer, primary thrombocytosis, primary macroglobulinemia, small cell lung cancer, ovarian serous cystadenocarcinoma, primary brain tumors, malignant pancreatic insulinoma, malignant carcinoid, premalignant skin lesions, prostate neuroendocrine cancer, testicular germ cell tumor, lymphomas, genitourinary tract cancer, malignant hypercalcemia, endometrial cancer, adrenal cortical cancer, Harding-Passey melanoma, juvenile melanoma, lentigo maligna melanoma, acral-lentiginous melanoma, amelanotic melanoma, benign juvenile melanoma, Cloudman's melanoma, S91 melanoma, nodular melanoma, subungual melanoma, and superficial spreading melanoma.

2. The method of claim 1, wherein the PRX3 inhibitor is thiostrepton.

3. The method of claim 1, wherein the cancer is a sarcoma.

4. The method of claim 3, wherein the sarcoma is angiosarcoma, dermatofibrosarcoma, epithelioid sarcoma, Ewing's sarcoma, fibrosarcoma, gastrointestinal stromal tumors (GIST), Kaposi sarcoma, leiomyosarcoma, liposarcomas, dedifferentiated liposarcoma, pleomorphic myxoid liposarcoma, malignant fibrous histiocytoma, undifferentiated pleomorphic sarcoma, neurofibrosarcoma, rhabdomyosarcoma (RMS), embryonal rhabdomyosarcoma, alveolar rhabdomyosarcoma, synovial sarcoma, alveolar soft part sarcoma, chondrosarcoma, desmoid sarcoma, malignant peripheral nerve sheath tumor (MPNST), uterine carcinosarcoma, myxofibrosarcoma, undifferentiated sarcoma NOS, INI-deficient soft tissue sarcoma NOS, malignant peripheral nerve sheath tumor, desmoplastic small round cell tumor, spindle cell/sclerosing rhabdomyosarcoma, undifferentiated spindle cell sarcoma, atypical teratoid/rhabdoid tumor, fibromatosis, inflammatory myofibroblastic tumor, infantile fibrosarcoma, sclerosing epithelioid fibrosarcoma, myofibromatosis, undifferentiated hepatic sarcoma, endometrial stromal sarcoma, lipoblastomatosis, sarcomatoid disease, melanocytic clear cell sarcoma (such as soft tissue melanoma), dermal pleomorphic sarcoma, or osteosarcoma.

5. The method of claim 1, wherein the cancer is a sarcomatoid carcinoma.

6. The method of claim 5, wherein the sarcomatoid carcinoma is associated with the subject's skin, bone, mouth, thyroid gland, breast, stomach, small intestine, liver, pancreas, uterus, prostate, bladder, kidneys, mediastinum, or lung.

7. The method of claim 1, further comprising administering an effective amount of a cancer chemotherapeutic agent.

8. The method of claim 7, wherein the cancer chemotherapeutic agent is selected from the group consisting of thiotepa, cyclophosphamide, busulfan, improsulfan, piposulfan, benzodopa, carboquone, meturedopa, uredopa, altretamine, triethylenemelamine, triethylenephosphoramide, triethylenethiophosphoramide, trimethylolmelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin; neocarzinostatin chromophore, aclacinomycins, actinomycin, authramycin, azaserine, bleomycins, cactinomycin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatrexate, demecolcine, diaziquone, eflornithine, elliptinium acetate, epothilone, etoglucid, gallium nitrate, hydroxyurea, lentinan, lonidamine, maytansine, ansamitocins, mitoguazone, mitoxantrone, mopidamol, nitracrine, pentostatin, phenamet, pirarubicin, losoxantrone, podophyllinic acid, 2-ethylhydrazide, procarbazine, PSK polysaccharide complex, razoxane, rhizoxin, sizofiran, spirogermanium, tenuazonic acid, triaziquone; 2,2′,2″-trichlorotriethylamine, trichothecene, T-2 toxin, verrucarin A, roridin A, anguidine, urethane, vindesine, dacarbazine, mannomustine, mitobronitol, mitolactol, pipobroman, gacytosine, arabinoside, cyclophosphamide, paclitaxel, docetaxel, chlorambucil, gemcitabine, 6-thioguanine, mercaptopurine, methotrexate, cisplatin, oxaliplatin, carboplatin, vinblastine, platinum, etoposide, ifosfamide, vincristine, vinorelbine, novantrone, teniposide, edatrexate, daunomycin, aminopterin, xeloda, ibandronate, irinotecan, RFS 2000, difluoromethylornithine, retinoic acid, and capecitabine.

9. A method of treating cancer in a subject in need thereof comprising:
(i) receiving information on the quantitative expression of a biomarker associated with epithelial-mesenchymal transition (EMT), wherein the biomarker is characteristic for a mesenchymal cell phenotype or for an epithelial cell phenotype, wherein the subject has an increased expression of the biomarker associated with EMT characteristic for a mesenchymal cell phenotype or wherein the subject has a decreased expression of the biomarker associated with EMT characteristic for an epithelial cell phenotype, and
(ii) administering to the subject an effective amount of a PRX3 inhibitor.

10. The method of claim 9, wherein the PRX3 inhibitor is thiostrepton.

11. The method of claim 9, wherein PRX3 inhibitor decreases the mesenchymal cell phenotype or increases the epithelial cell phenotype.

12. The method of claim 9, further comprising:
(iii) receiving information on the quantitative expression of the biomarker associated with EMT after administering the PRX3 inhibitor, and
(iv) administering an effective amount of a cancer chemotherapeutic agent if the biomarker characteristic for a mesenchymal cell phenotype is decreased or if the biomarker characteristic for an epithelial cell phenotype is increased.

13. The method of claim 12, wherein the cancer chemotherapeutic agent is selected from the group consisting of thiotepa, cyclophosphamide, busulfan, improsulfan, piposulfan, benzodopa, carboquone, meturedopa, uredopa, altretamine, triethylenemelamine, triethylenephosphoramide, triethylenethiophosphoramide, trimethylolmelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin; neocarzinostatin chromophore, aclacinomycins, actinomycin, authramycin, azaserine, bleomycins, cactinomycin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatrexate, demecolcine, diaziquone, eflornithine, elliptinium acetate, epothilone, etoglucid, gallium nitrate, hydroxyurea, lentinan, lonidamine, maytansine, ansamitocins, mitoguazone, mitoxantrone, mopidamol, nitracrine, pentostatin, phenamet, pirarubicin, losoxantrone, podophyllinic acid, 2-ethylhydrazide, procarbazine, PSK polysaccharide complex, razoxane, rhizoxin, sizofiran, spirogermanium, tenuazonic acid, triaziquone; 2,2′,2″-trichlorotriethylamine, trichothecene, T-2 toxin, verrucarin A, roridin A, anguidine, urethane, vindesine, dacarbazine, mannomustine, mitobronitol, mitolactol, pipobroman, gacytosine, arabinoside, cyclophosphamide, paclitaxel, docetaxel, chlorambucil, gemcitabine, 6-thioguanine, mercaptopurine, methotrexate, cisplatin, oxaliplatin, carboplatin, vinblastine, platinum, etoposide, ifosfamide, vincristine, vinorelbine, novantrone, teniposide, edatrexate, daunomycin, aminopterin, xeloda, ibandronate, irinotecan, RFS 2000, difluoromethylornithine, retinoic acid, and capecitabine.

14. The method of claim 9, wherein the cancer is selected from the group consisting of sarcoma, sarcomatoid carcinoma, thymoma, glioblastoma multiforme, adrenocortical cancer, uterine carcinosarcoma, uterine corpus endometrioid carcinoma, kidney clear cell carcinoma, mesothelioma, brain lower grade glioma, pheochromocytoma and paraganglioma, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, KRAS-mutant melanoma, renal cell carcinoma of kidney, kidney papillary cell carcinoma, reserve cell carcinoma, Hodgkin's Disease, Non-Hodgkin's Lymphoma, multiple myeloma, neuroblastoma, breast cancer, triple-negative breast cancer, ovarian cancer, lung cancer, primary thrombocytosis, primary macroglobulinemia, small cell lung cancer, ovarian serous cystadenocarcinoma, primary brain tumors, stomach cancer, colon cancer, malignant pancreatic insulinoma, malignant carcinoid, premalignant skin lesions, testicular cancer, prostate neuroendocrine cancer, testicular germ cell tumor, lymphomas, thyroid carcinoma, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungual melanoma, and superficial spreading melanoma.

15. The method of claim 14, wherein the mesothelioma is sarcomatoid mesotheliomas(S) or epithelioid mesotheliomas (E).

16. The method of claim 9, wherein the cancer is a sarcoma.

17. The method of claim 16, wherein the sarcoma is angiosarcoma, dermatofibrosarcoma, epithelioid sarcoma, Ewing's sarcoma, fibrosarcoma, gastrointestinal stromal tumors (GIST), Kaposi sarcoma, leiomyosarcoma, liposarcomas, dedifferentiated liposarcoma, pleomorphic myxoid liposarcoma, malignant fibrous histiocytoma, undifferentiated pleomorphic sarcoma, neurofibrosarcoma, rhabdomyosarcoma (RMS), embryonal rhabdomyosarcoma, alveolar rhabdomyosarcoma, synovial sarcoma, alveolar soft part sarcoma, chondrosarcoma, desmoid sarcoma, malignant peripheral nerve sheath tumor (MPNST), uterine carcinosarcoma, myxofibrosarcoma, undifferentiated sarcoma NOS, INI-deficient soft tissue sarcoma NOS, malignant peripheral nerve sheath tumor, desmoplastic small round cell tumor, spindle cell/sclerosing rhabdomyosarcoma, undifferentiated spindle cell sarcoma, atypical teratoid/rhabdoid tumor, fibromatosis, inflammatory myofibroblastic tumor, infantile fibrosarcoma, sclerosing epithelioid fibrosarcoma, myofibromatosis, undifferentiated hepatic sarcoma, endometrial stromal sarcoma, lipoblastomatosis, sarcomatoid disease, melanocytic clear cell sarcoma (such as soft tissue melanoma), dermal pleomorphic sarcoma, or osteosarcoma.

18. The method of claim 9, wherein the cancer is a sarcomatoid carcinoma.

19. The method of claim 18, wherein the sarcomatoid carcinoma is associated with the subject's skin, bone, mouth, thyroid gland, breast, stomach, small intestine, liver, pancreas, uterus, prostate, bladder, kidneys, mediastinum, or lung.

20. A method of treating cancer in a subject in need thereof comprising administering to the subject an effective amount of a PRX3 inhibitor and a SLC7A11 inhibitor.

21. The method of claim 20, wherein the PRX3 inhibitor is thiostrepton.

22-31. (canceled)

32. A method of treating cancer in a subject in need thereof comprising:
(i) receiving information on the quantitative expression of SLC7A11 protein in cancer tissue, and
(ii) administering to the subject an effective amount of a PRX3 inhibitor if the quantitative expression of SLC7A11 protein in cancer tissue is lower than or similar to the quantitative expression in normal or healthy tissue; or administering to the subject an effective amount of a PRX3 inhibitor and a SLC7A11 inhibitor if the quantitative expression of SLC7A11 protein in cancer tissue is higher than the quantitative expression in normal or healthy tissue.

33. The method of claim 32, wherein the PRX3 inhibitor is thiostrepton.

34-43. (canceled)
